(19) United States
(12) Patent Application Publication
(10) Pub. No.: US 2016/0320371 A1
(43) Pub. Date: Nov. 3, 2016

(54) ONLINE DIAGNOSTIC SYSTEM WITH VIRTUAL MEDICAL ASSISTANCE FOR THE DIAGNOSED DISEASE UTILIZING NANO-DEVICES AND QUANTUM CLOUD COMPUTING

(71) Applicant: Fred Abraham, Tempe, AZ (US)

(72) Inventor: Fred Abraham, Tempe, AZ (US)

(21) Appl. No.: 15/074,749

(22) Filed: Mar. 18, 2016

Related U.S. Application Data
(60) Provisional application No. 62/135,272, filed on Mar. 19, 2015.

(51) Int. Cl. G01N 33/53; G06F 19/00; G01N 35/00; G06Q 20/18; G01N 1/00

(52) U.S. Cl. CPC G01N 33/5308; G06Q 20/18; G01N 1/00; G01N 35/00871; G06F 19/366; G01N 2001/002; G01N 2035/00881; G01N 2035/00891

(57) ABSTRACT

The present invention relates to implementing an online diagnostic system for diagnosing the sample received from a client. The system is connected with an electrode through a Bluetooth low energy communication channel to receive the sample from one or more subscribed client(s) and the system is configured to transmit the received sample for further diagnosis of the sample. Additionally, the system can be supported with a diagnostic kiosk integrated with a dual purpose chip/imbedded Microfluidic chip (MFC) to collect the test sample. Further, the transmitted sample is diagnosed in the cloud by using nano technology and through the implementation of the cloud computation technology. Further, the application is configured to send back the sample diagnosis result to the electronic device from which the client sample was collected or through a diagnostic kiosk LED display to provide a virtual medical assistance for the clients diagnosed with a disease.

### CROSS-REFERENCE TO RELATED APPLICATION

This application claims priority to the U.S. Provisional Patent application No. 62135272 filed in the United States Patent and Trademark Office on Mar. 19, 2015, entitled “Online Diagnostic System”. The specification of the above referenced patent application is incorporated herein by reference in its entirety

### FIELD OF THE INVENTION

The present invention generally relates to implementing a diagnostic system and more particularly relates to an online diagnostic system that receives the client's sample through an electrode connected to the electronic device or from a diagnostic kiosk, which uses a disposable dual purpose pin/prick embedded Microfluidic chip (MFC) and utilizes nano technology to analyze the client's sample. Further, the client's sample intensities collected through the electrode is processed through the cloud computing technology. The diagnosed sample result is transmitted back to the electronic device and the system provides the user with a virtual medical assistance for treating the diagnosed disease.

### BACKGROUND OF THE INVENTION

The concept of vending machine style diagnosis machine and mobile phone/iphone device having an accessory (chip or probe or any detection means) to diagnose the sample is already known in the art.

In some of the prior-art, an automated vending machine is described where the user can put their biological sample in the allotted slot to further investigate suspected diseases caused by different categories of microorganisms.

Another existing prior-art, describes a vending machine that performs nucleic acid extraction from the specimen and produces a genetic assay from the specimen and produces a disease diagnoses by implementing a method for an automated genetic assay diagnostic instrument.

Further, another prior-art teaches to detect targets such as HIV, HBV, HCV and sexually transmitted diseases. The prior-art describes the system and method used to detect and diagnose molecular diagnostic targets arising in the fields of oncology, cardiovascular, identity testing and prenatal screening. The dispensing activator system used to detect and diagnose molecular diagnostic targets comprises of a vending machine, an automated teller machine, and a kiosk.

Another existing prior-art teaches a diagnostic system used to analyze gonorrhea. Automated dispensing assembly is configured to identify medication corresponding to the prescribed medication in the prescription information in its stored location and to dispense the medication using techniques known in the art of medication dispensing and in vending machine technology.

Based on the above discussed prior-art, the existing diagnostic systems have relatively complex assembly structure with implementation of specialized methods for diagnosing the sample. Further, with the advent of numerous electronic devices and gadgets in the industry, the assembly structure can get more complicated along with the implementation of the method used for diagnosing the sample. Further, with the evolution of nano technology having an impact on various domains and with the advent of wireless communication channels, analysis of the client's sample can be more precise, simpler, and faster.

Accordingly, there exists a need for a simple mode of collecting the client sample and performing diagnosis of the collected sample accurately by using the nano technology.

### SUMMARY OF THE INVENTION

The present invention relates to implementing an online diagnostic system, wherein the system comprises of an electrode that is inserted into a container containing the client sample, which is connected to an electronic device through a Bluetooth low energy communication channel for receiving one or more client sample intensities, or the system is integrated with a disposable dual purpose pin/prick embedded with the Microfluidic chip (MFC) diagnostic kiosk for receiving the client sample intensities. Further, the system transmits the received sample intensities to a cloud computing technology for diagnosing the client's sample, and the system displays the diagnosis result on the electronic device or the Light Emitting Diode (LED) screen on the diagnostic screen, within a short interval of time. After diagnosing the client's sample, the online diagnostic system provides a virtual medical assistance for the diagnosed disease.

### BRIEF DESCRIPTION OF DRAWINGS

FIG. 1 illustrates a mobile phone connected with an electrode to receive the client's sample and to process the client sample by implementing the cloud computing technology.

FIG. 2 illustrates an overview of the system implemented to perform online sample diagnosis.

FIG. 3 illustrates an overview of an electrode inserted into a container for collecting the client's sample.

FIG. 4 illustrates a flow-chart that explains the process of online sample diagnosis.

FIG. 5 illustrates an overview of components used to implement the online sample diagnosis system.

FIGS. 6a and 6b illustrate a cross-sectional overview of the dual purpose pin/prick with embedded microfluidic chip used for drawing testing samples from the client(s).

FIGS. 7a, 7b, 7c, 7d, and 7e illustrate an overview of a diagnostic kiosk with a disposable dual purpose pin/prick embedded with the Microfluidic chip (MFC) for receiving and analyzing the client sample intensities.

FIG. 8 illustrates exemplary screen images of mobile application user interface.

### FIGURE DESCRIPTION

100—An online diagnostic system overview
101—A mobile phone connected with an electrode
102—An electrode connected to the mobile device through a Bluetooth low energy communication channel
200—Depicts the mobile devices connected to the cloud where the client sample is analyzed
201—Depicts a cloud where the collected sample is transmitted for diagnosis by implementing a cloud computing technology
202a, 202b, and 202c—Depicts a plurality of mobile devices connected within the network
203—Client sample collected through the electrode connected to the mobile device
300—A container used for collecting the client &ample through an electrode
301—An electrode connected to the lid of the container
302—Depicts a black opaque container used to collect the client sample
400—A flow-chart that explains the process of online sample diagnosis
500—A system overview of components used for implementing the method
501—A Sample collection module
502—A Diagnosis module
503—A Subscription module
504—A Controlling module
505—A Display module
600—An overview of the dual purpose pin/prick and embedded Microfluidic chip dual purpose pin/prick with embedded microfluidic chip
601—A pair of piercing tips provided at the front of the dual purpose pin/prick and embedded microfluidic chipt dual purpose pin/prick with embedded microfluidic chip
602—A sample collection container
603—A sample flowing through the container channel
700—An overview of a diagnostic kiosk
700a—A disposable Microfluidic chip (MFC) inserted in the diagnostic kiosk
700b—A Light Emitting Diode (LED) display on the diagnostic kiosk
700c—An interface that allows the user to select the test to be performed
700d—A slot used for inserting the MIC chip in the diagnostic kiosk
701—Illustrates an internal working of the diagnostic kiosk
702—A receiver provided in the diagnostic kiosk to receive the client sample
703—A nano-device solution collected through a diagnostic kiosk channel
704—A chamber through which the nano-device solution flows
705—A robot arm directing the flow of the nano-device solution
706—Injecting the nano-device solution into a 3D MFC chip cavity dual purpose pin/prick with embedded microfluidic chip
706a—Attaching a injection syringe to MFC chip dual purpose pin/prick with embedded microfluidic chip to inject with nano-device solution
707—A 3D MFC chip cavity used for analyzing the client's sample
800, 801—Screen Images of mobile application user interface

### DETAILED DESCRIPTION OF THE INVENTION

The following detailed description of the preferred embodiments presents a description of certain specific embodiments to assist in understanding the claims. However, the present invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be evident to one of ordinary skill in the art that the present invention may be practiced without these specific details.

In an embodiment, an electronic device that can be used to connect an antenna electrode through a Bluetooth low energy communication for receiving the client's sample, wherein the electronic device includes but not limited to a mobile phone, a smart phone, an ipad, a laptop, a tablet, a personal computer, or any other communication device.

In an embodiment, the electronic device is configured to support an application that is integrated with a dual purpose pin/prick dual purpose pin/prick with embedded microfluidic chip embedded with a Microfluidic chip (MFC) for drawing the sample, and further the collected sample is analyzed through a diagnostic kiosk based on the displacement intensities measured between the nano-device solution and the sample drawn from the client, which is further processed through a cloud computing technology.

In an embodiment, the nano-device solution gets injected into the chip, after the user pin-pricks themselves utilizing the dual purpose chip/imbedded Microfluidic chip (MFC) and inserts it into the slot as the solution is injected and the solution permeates into the cavity. Further, the nano-device solution injected into the cavity meets the client's sample and the coupling takes places, which excites the nano-device solution as higher intensities are created within the phonon waves. Further, the system measures the displacement of intensities between the nano-device solution and the client's sample, and the coupling takes place if the antigen or any required component is present within the client's sample.

In an embodiment, the term client can refer to a human-being, an animal, or any other living being from which the sample is extracted for diagnosis purpose and the client must have subscribed to the system for getting the sample diagnosed. Further, the sample diagnosis can be performed on a regular basis such as monthly, weekly, quarterly, half-yearly, or the like based on the type of subscription opted by the client.

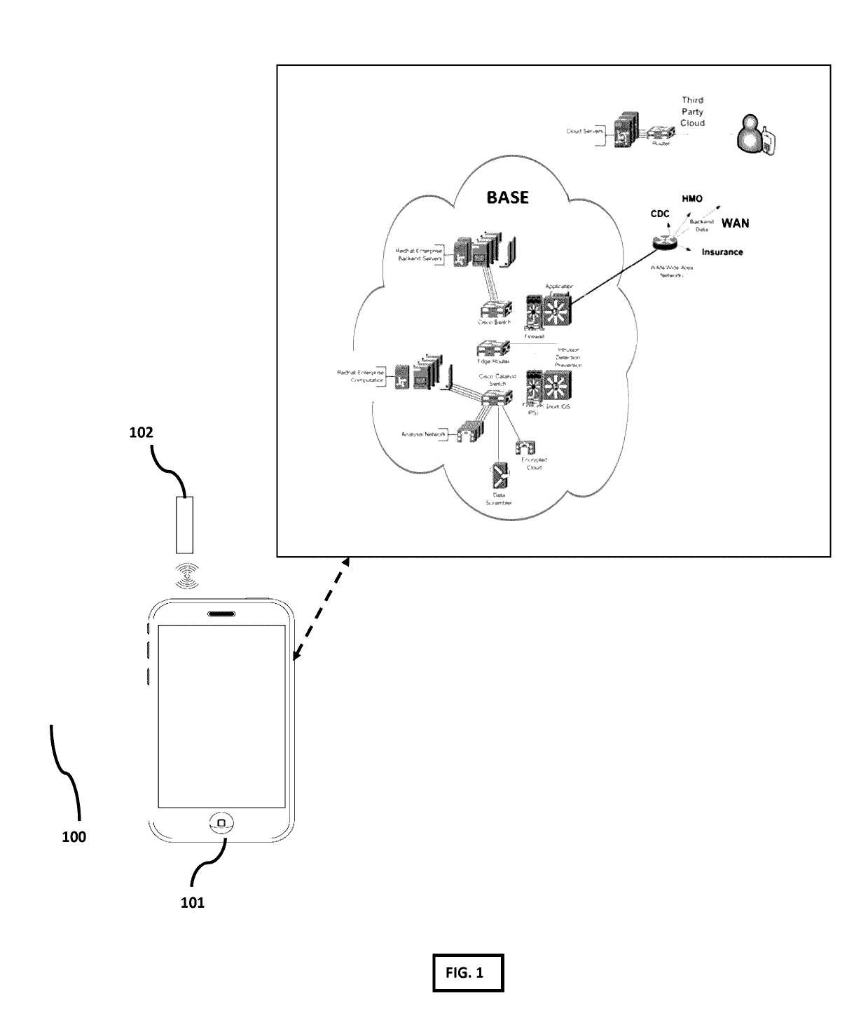

Referring to FIG. 1, illustrates a mobile phone 101 connected with an electrode 102 to receive the client's sample and to process the client sample by adopting the cloud computing technology. In an embodiment, the electrode 102 used to collect the sample from the client is placed in a container inserted from the top of the container lid, which is connected to the electronic device through a Bluetooth low energy communication channel. Further, as depicted in the figure, the client sample collected through the electrode can be transmitted to a cloud network to analyze the client sample.

In another embodiment, a diagnostic kiosk with a dual purpose pin/prick embedded with a Microfluidic chip (MFC) can be used to collect client's sample and further can be used to analyze the components/disease of a client sample with a nano-device solution injected within Microfluidic chip (MFC) that shows the coupling of the nano device solution with the client's sample when a potential host virus/component/antigen is found in the client sample.

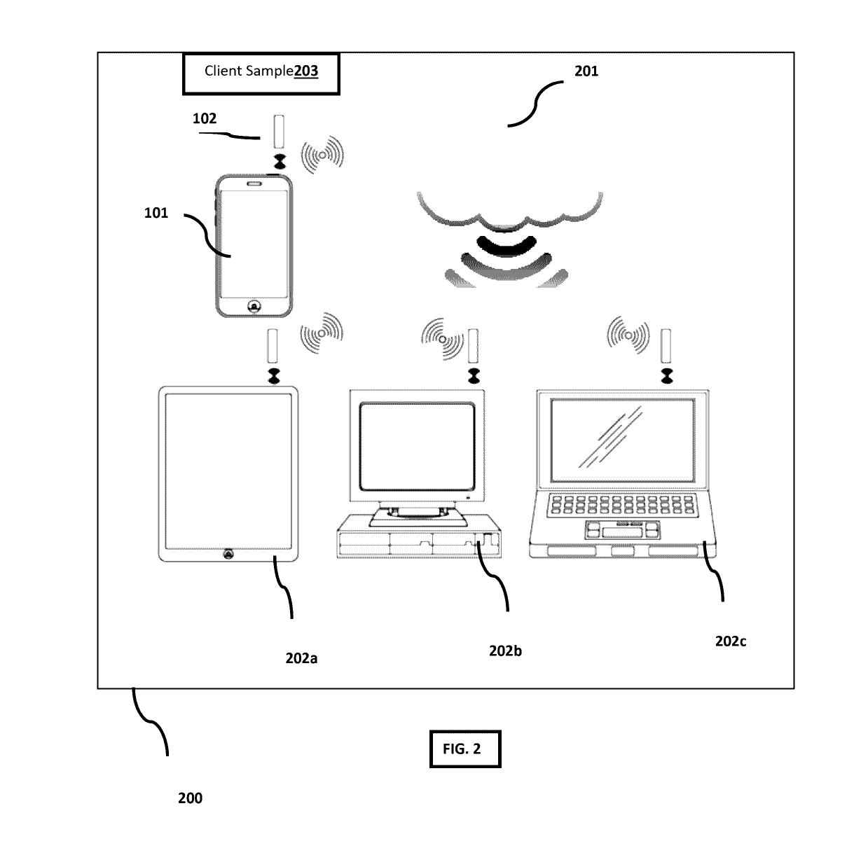

FIG. 2 illustrates an overview of the system 200 implemented to perform sample diagnosis. In an embodiment, the system 200 comprises of: an electrode 102 inserted into a client sample collecting container 302 and connected to the electronic devices 202a, 202b, and 202c, through a Bluetooth low energy communication channel and the electrode 102 is configured to receive one or more client's sample for diagnosis purpose. The client's sample can include a blood sample, urine sample, a fluid sample or any other sample that can be used for diagnosis purpose. Further, an application is provided for transmitting the received sample to a lab 201 for diagnosing the sample received at the electrode 102. In an embodiment, the sample diagnosis is performed in the cloud 201 by analyzing the collected sample. For example, the sample diagnosis can be used for determining the chemical, biological, or molecular composition of the sample. As another example, the sample diagnosis can be used to determine the type of disease incurred by the client based on the client's sample received at the electrode 102. In an embodiment, the application is configured to receive the diagnosis result at one or more electronic devices 202a, 202b, and 202c through which the client's sample is collected. Further, the diagnosis result is displayed on the electronic devices 202a, 202b, and 202c within a short interval of time.

In another embodiment, the electronic devices 202a, 202b, and 202c is configured with a mobile application that allows a user to draw sample through a diagnostic kiosk 700 integrated with a disposable dual purpose pin/prick embedded with the MFC chip. Further, the diagnostic kiosk 700 is configured to perform sample analysis by implementing a nano technology and further processing the client's sample through a cloud computation technology. Further, the diagnostic kiosk 700 is configured to display the output result of the processed analysis on the electronic devices 202a, 202b, and 202c and provides assistance with a virtual medical practitioner to suggest medication or to refer a specialist for providing online treatment for the diagnosed disease.

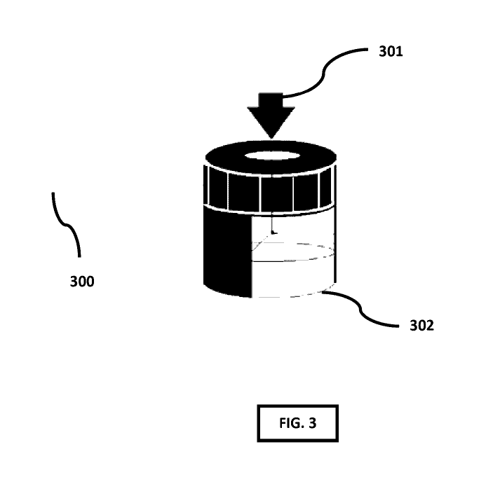

FIG. 3 illustrates an overview of an electrode inserted into a container for collecting the client's sample. In an embodiment, the electrode 102 is inserted from the top opening 301 of the lid of the container and the container 302 is encased with a dark opaque plastic material to avoid ambient light. Further, the collected client sample is transmitted through the electrode 301 for processing the client sample through the cloud computation technology.

FIG. 4 illustrates a flow-chart 400 that explains the process of online sample diagnosis. Initially, at step 301, a user is allowed to register with the application installed on the electronic devices 202a, 202b, and 202c for performing an online diagnosis of the sample collected through the electrode 102 or through the diagnostic kiosk 700. In an embodiment, at step 402, the electrode 102 connected through the electronic device is configured to collect the client sample. In another embodiment, the client sample can be collected through the diagnostic kiosk 700. A disposable dual purpose pin/prick embedded with the Microfluidic chip (MFC) is used for collecting the testing sample of the client(s). Further, at step 403, the client sample is collected in the container 300 along with the nano-device solution. For example, the electronic device 202b can be configured to collect blood sample from client A for diagnosing the blood composition. At step 404, the electrode 102 connected to the mobile device transmits the collected sample to the cloud 201 or implements the cloud computing technology for sample diagnosis. For example, client A's blood sample can be diagnosed with diabetes. At step 405, the electrode 102 connected to be mobile device from where the sample is collected is configured to receive the diagnosed result within a short interval. Based on the diagnosed result, the application is configured to provide a virtual assistance for the client. For example; the electronic device 202b can receive the diagnosed result of client A after the blood sample is analyzed in the cloud 201 or through cloud computation. Further, at step 406, the application frequently monitors for receiving the client sample.

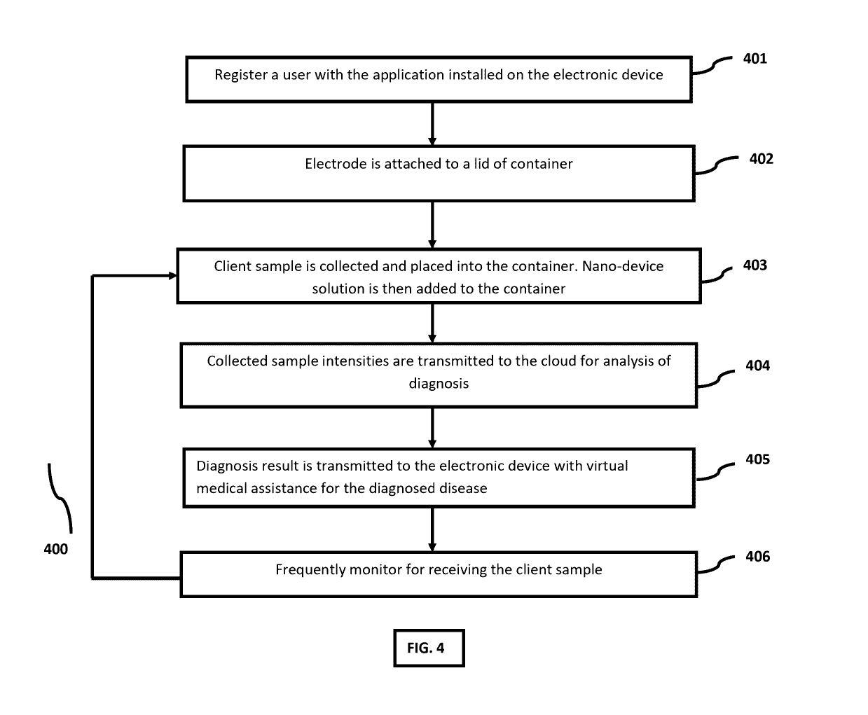

FIG. 5 illustrates an overview of components 500 used to implement the online diagnosis of the client sample. In an embodiment, the system 500 comprises of the following components: a Sample collection module 501, a Diagnosis module 502, a Subscription module 503, a Controlling module 504, and a Display module 505. In an embodiment, the Sample collection module 501 is configured to collect client samples from the electrode 102 connected to the electronic device 101 or through a diagnostic kiosk with a disposable dual purpose pin/prick embedded with an MFC. In an embodiment, the Diagnosis module 502 is configured to diagnose the client sample collected from the electrode 102 and the diagnosis of the client sample is performed in a cloud 201 by implementing any of the existing diagnosis method or process by implementing a cloud computing technology. In another embodiment, the Diagnosis module 502 is configured to allow the diagnostic kiosk 700 with an embedded MFC to process or analyze the collected client sample. In an embodiment, the Subscription module 503 is configured to receive subscription from one or more clients for subscribing to the online sample diagnosis system 200. The client can opt to subscribe for a monthly, yearly, quarterly, weekly, or the like based on the frequency of sample diagnosis required by the client. In an embodiment, the Controlling module 504 can be configured to transmit the sample data and the result data across the modules used to implement the system 200. In an embodiment, the Display module 505 is configured to display the diagnosis results on the electronic device 101 or through a Light Emitting Diode (LED) display area 700b for providing a virtual medical assistance for the diagnosed disease of the user.

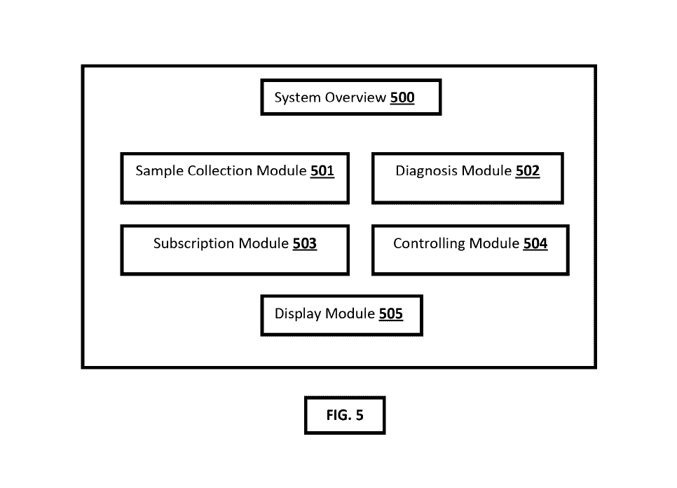

FIGS. 6a and 6b illustrate a cross-sectional overview of the dual purpose pin/prick with embedded microfluidic chip used for drawing testing samples from the client(s). In an embodiment, the front-end of the dual purpose pin/prick with embedded microfluidic chip is provided with piercing tips 601 for drawing the samples from an individual. Further, as the samples are drawn from the piercing tips 601, the sample is collected in a blood/sample holding reservoir 602 and later the sample flows through the dual purpose pin/prick with embedded microfluidic chip channel 603, where the blood sample is prepared for processing/analyzing through cloud computation. In an embodiment, the nano-device solution can be injected into the dual purpose pin/prick with embedded microfluidic chip channel 603 for analyzing the client sample in the dual purpose pin/prick with embedded microfluidic chip. In another embodiment, an MFC chip 700a filled with a nano-device solution can be fixed to the dual purpose pin/prick with embedded microfluidic chip channel 603 for analyzing the client sample.

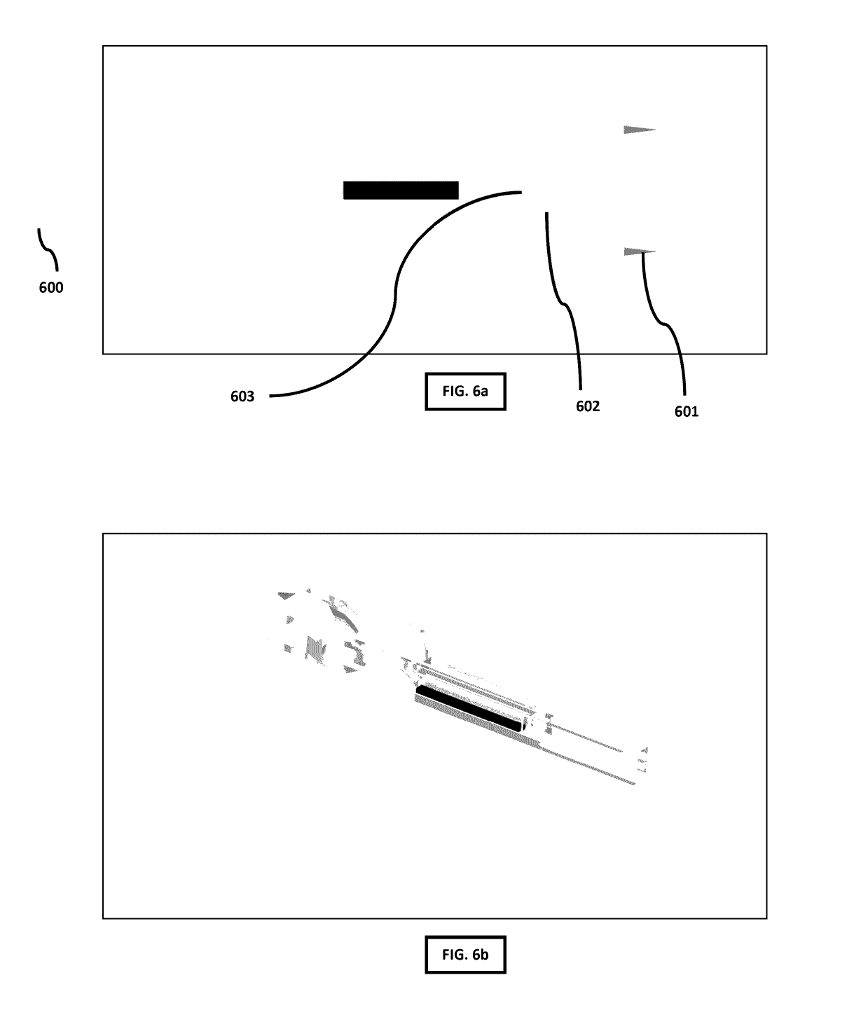

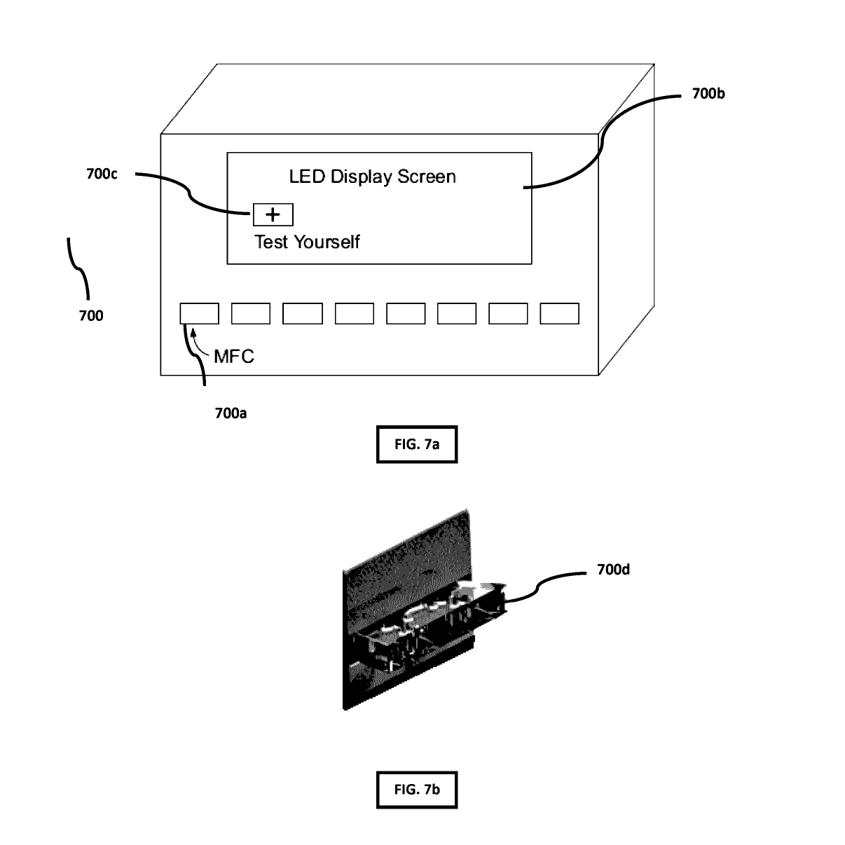

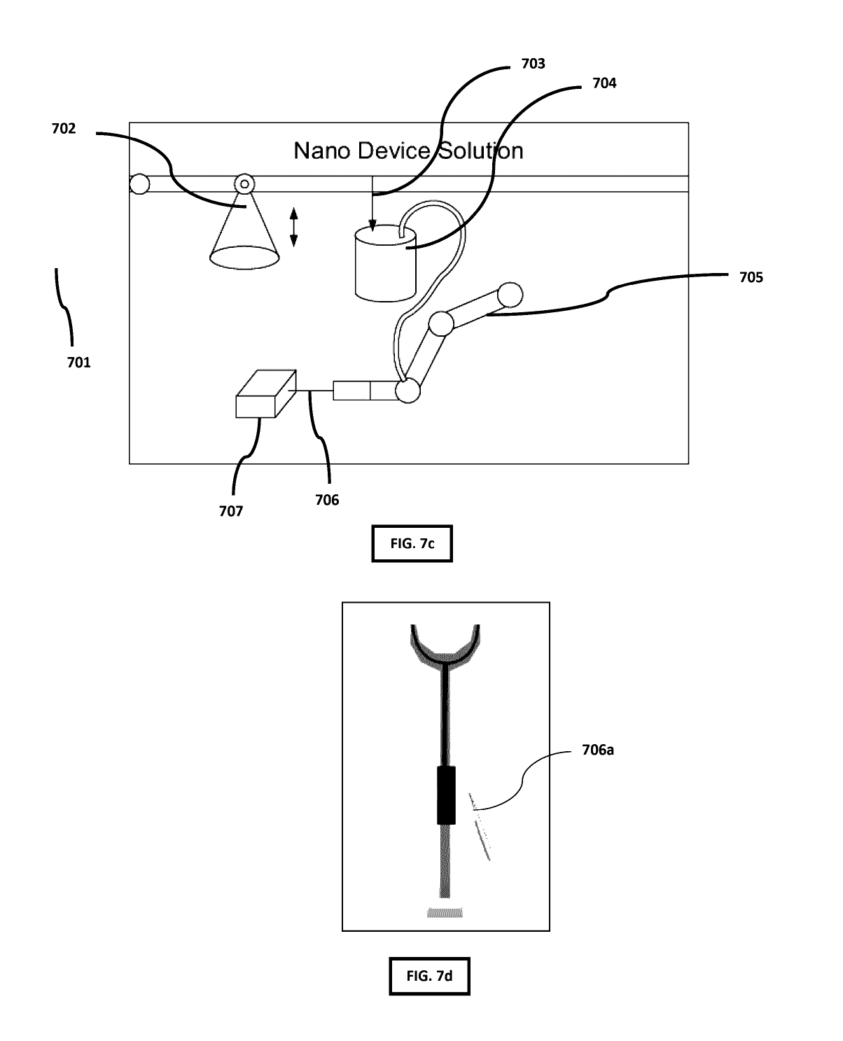

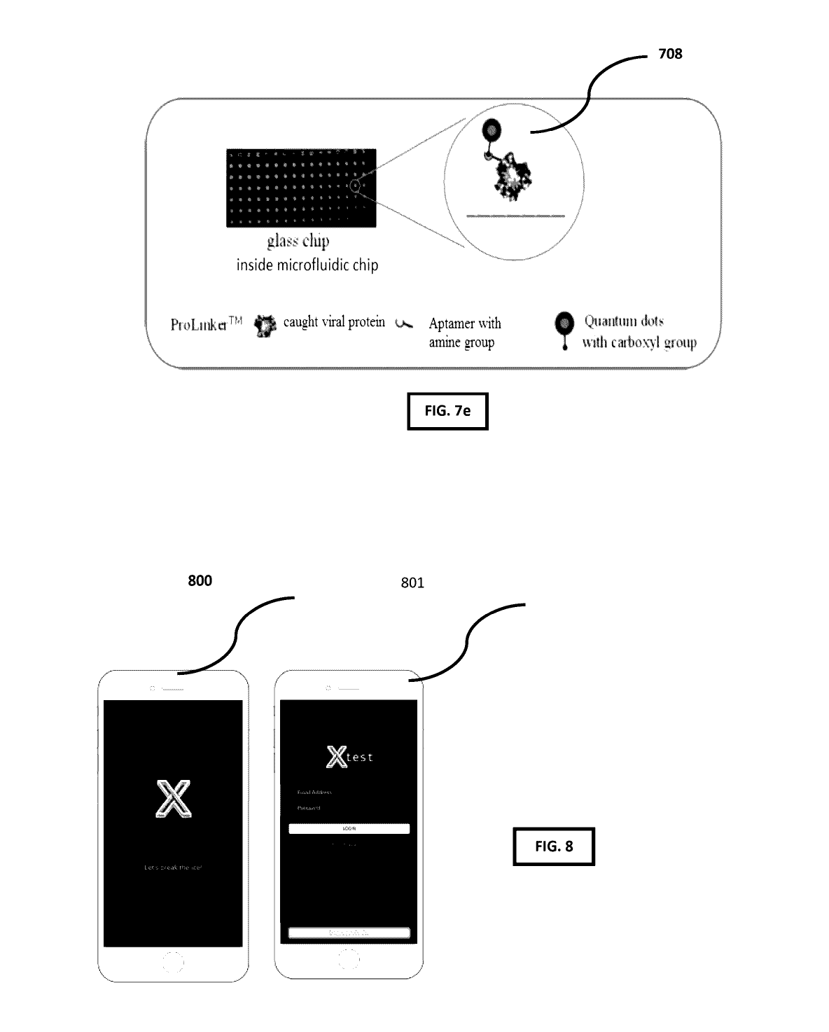

FIGS. 7a, 7b, 7c, 7d, and 7e illustrate an overview of a diagnostic kiosk with a disposable dual purpose pin/prick embedded with the MFC for receiving and analyzing the client sample intensities. As depicted in the FIG. 7a, the diagnostic kiosk 700 is provided with an LED display 700b to indicate the availability of an MFC chip 700a inserted within the kiosk 700.

In an embodiment, the diagnostic kiosk 700 is configured to allow 8 users to collect and analyze the sample at any instance and is configured to store 150 containers of nano-device solution for testing each infection and disease associated with the client sample.

In an embodiment, a user interface 700c is provided on the user interface for allowing the client/user to select the test to be conducted on the collected sample.

As depicted in FIG. 7b, the slot 700d is for dual-prong chip to get inserted into, wherein the chip does not have nano-device solution in the MFC chip and gets injected after the chip is inserted into the slot and based on the type of test to be conducted.

Further, FIG. 7c depicts the internal working of a disposable MFC chip 701 inserted within the diagnostic kiosk 700. In an embodiment, the receiver 702 receives the client's sample for diagnosis purpose, and the receiver 702 can be extended either vertically or horizontally to collect the sample. Further, the nano-device solution 703 is collected through the chamber 704, and as the nano-device solution 703 flows into the robotic arm 705 through a connector the solution gets injected 706 into the MFC chip cavity 707 or to an MFC chip 707 connected to the dual purpose pin/prick with embedded microfluidic chip as depicted in FIG. 7d for performing the analysis on the client's sample. In an embodiment, as the nano-device solution meets the client's sample in a 3D MFC chip cavity 707, the coupling takes place when the nano-device solution excites due to higher intensities created within the phonon waves due to the presence of the antigen in the client's sample. FIG. 7e, depicts the coupling effect that takes places in a 3D MFC chip cavity 707 when the nano-device solution excites due to higher intensities created within the phonon waves due to the presence of the antigen in the client's sample.

The kiosk 700 is plugged into an electric outlet and will be connected through the interne. Further, the disposable MFC chips 700a can be associated with a storage unit for collecting the used chips, wherein the storage unit can be a hazardous material bin built into the kiosk 700.

In an embodiment, the kiosk 700 can be configured to dispense the MFC after the test is completed. Further, the MFC 700a can either be ejected from the slot or thrown into the hazardous bin as soon as the test is completed. Optionally, bottom of the kiosk 700 can be used to collect the used MFC 700a.

Further, the analysis result is transmitted to a cloud database for implementing a cloud computing technology to further process the client's sample.

FIG. 8 illustrates exemplary screen images 800, 801 of the mobile application user interface.

1. An online diagnostic system used for diagnosing at least one client's sample by implementing a nano technology through a mobile application, wherein said system is configured to: receive a subscription from said at least one client for subscribing to said online diagnostic system allow said at least one client to select at least one test for testing said at least one client's sample through said mobile application; receive said at least one client sample through an electrode connected to an electronic device through a Bluetooth low energy communication channel; optionally receive said at least one client's sample through a diagnostic kiosk integrated with a dual purpose chip/imbedded Microfluidic chip (MFC); transmit the received said at least one client's sample to a diagnosis center for diagnosing said at least one client's sample; optionally collect the nano-device solution through the dual purpose chip/imbedded MFC; implement said nano technology for diagnosing said at least one client's sample by measuring the displacement of intensities between the nano-device solution and said at least one client's sample to determine the presence of at least one antigen in said at least one client's sample; implement a cloud computing technology for further processing of said at least one client sample by transmitting said at least client sample to a cloud database; display the diagnosis result on said electronic device after said at least one client's sample is diagnosed within a short interval of time; and provide a virtual medical assistance for said at least one client based on the diagnosed result.

2. The system as claimed in claim 1, wherein said system is configured to receive the nano-device solution and said at least one client's sample through said diagnostic kiosk into a Microfluidic chip (MFC) for measuring the displacement intensities between the nano-device solution and said at least one client's sample to determine the presence of at least one antigen in said at least one client's sample.

3. The system as claimed in claim 2, wherein said system is configured to allow 8 users to collect the sample and perform analysis at any instance of time.

4. The system as claimed in claim 2, wherein said system is configured to store up to 150 nano-device solutions to facilitate different type of tests on the collected sample.

5. The system as claimed in claim 2, wherein the system is configured to provide a coupling effect when higher displacement intensities are created within the phonon waves as the presence of said at least one antigen is determined in said at least one client's sample.

6. A method implemented in an online diagnostic system for diagnosing at least one client's sample by implementing a nano technology through a mobile application, wherein said method comprises of: receiving a subscription from said at least one client for subscribing said at least one client with said online diagnostic system; allowing said at least one client to select at least one test for testing said at least one client's sample through said mobile application; receiving said at least one client' sample through an antenna electrode connected to an electronic device through a Bluetooth low energy communication channel: optionally receiving said at least one client's sample through a diagnostic kiosk integrated with a dual purpose chip/imbedded Microfluidic chip (MFC); transmitting the received said at least one client's sample to a diagnosis center for diagnosing said at least one client's sample;
optionally collect the nano-device solution through the dual purpose chip/imbedded MFC; implementing said nano technology for diagnosing said at least one client's sample by measuring the displacement of intensities between the nano-device solution and said at least one client's sample to determine the presence of at least one antigen in said at least one client's sample; implementing a cloud computing technology for further processing of said at least one client sample by transmitting said at least client sample to a cloud database; displaying the diagnosis result on said electronic device after said at least one client's sample is diagnosed within a short interval of time; and providing a virtual medical assistance for said at least one client based on the diagnosed result.

7. The method as claimed in claim 6, wherein the method receives the nano-device solution and said at least one client's sample through said diagnostic kiosk into a Microfluidic chip (MFC) for measuring the displacement intensities between the nano-device solution and said at least one client's sample to determine the presence of at least one antigen in said at least one client's sample.

8. The method as claimed in claim 7, wherein the method provides a coupling effect when higher displacement intensities are created within the phonon waves as the presence of said at least one antigen is determined in said at least one client's sample.

9. The method as claimed in claim 7, wherein said method allows 8 users to collect the sample and perform analysis at any instance of time.

10. The method as claimed in claim 7, wherein said method stores up to 150 nano-device solution to facilitate different type of tests on the collected sample.

11. The algorithm set and all of its independent or as a collective as described in the description section 38, utilization of classical and quantum physics to define the electrostatics intensities within a dynamic system like the human body and its particulates, internally or externally that may be affecting the system.
